(19) United States
(12) Patent Application Publication
(10) Pub. No.: US 2020/0185064 A1
(43) Pub. Date: Jun. 11, 2020

(54) METHOD FOR PREDICTING THE CROSS-RECOGNITION OF TARGETS BY DIFFERENT ANTIBODIES

(71) Applicants: INSTITUT NATIONAL DE LA RECHERCHE AGRONOMIQUE, PARIS (FR); CENTRE NATIONAL DE LA RECHERCHE SCIENTIFIQUE, PARIS (FR)

(72) Inventors: Thomas BOURQUARD, HOUSTON, TX (US); Astrid MUSNIER, TOURS (FR); Anne POUPON, MAZANGE (FR); Pascale CREPIEUX, SAINT CYR SUR LOIRE (FR); Eric REITER, NOUZILLY (FR); Gilles BRUNEAU, REUGNY (FR)

(21) Appl. No.: 16/349,325

(22) PCT Filed: Nov. 13, 2017
(86) PCT No.: PCT/FR2017/053083

§ 371 Date: May 13, 2019

(30) Foreign Application Priority Data
Nov. 14, 2016 (FR) 16 60940

(51) Int. Cl. G16B 50/30; G16B 15/20; G16B 30/00; C07K 16/18

(52) U.S. Cl. CPC G16B 50/30; G16B 15/20; B82Y 5/00; C07K 16/18; C07K 2317/565; G16B 30/00

(57) ABSTRACT

The present invention relates to an in silico method for predicting the ability of an antibody to recognize the epitope of another antibody, based on a measure of similarity of antibody sequence and structure.

### FIELD OF THE INVENTION

The present invention relates to the field of biology, in particular of antibodies.

### BACKGROUND OF THE INVENTION

The conventional methods for developing an antibody consist in immunizing an animal, then in recovering the animal's serum in order to obtain polyclonal antibodies or, in order to obtain monoclonal antibodies, in producing hybridomas from the animal's plasmocytes, or else in screening a phage library prepared from mRNA from the plasmocytes of the immunized animal (phage display).

The antibodies obtained in this way are subsequently tested in vitro, then in animals, then finally in humans. The development of such a therapeutic antibody costs several millions of euros and takes several years.

Thus, it would be extremely useful to be able to identify the potential cross-reactions of the antibodies in order to anticipate the possible side effects and toxicity thereof.

On the other hand, when the development of a therapeutic antibody ends at an impasse due to unfavorable physicochemical properties of the antibody, for example low solubility, poor stability, toxicity, or difficulty of production, it would be desirable to have a quicker alternative than restarting the development of the antibody from the beginning.

There are methods for modeling the 3D structure of antibodies. However, the methods described are generally complicated to implement and none of them make it possible to predict cross-recognition of targets between several antibodies.

This thus gives rise to a need for a method making it possible to identify, among known antibodies, those which are capable of recognizing the same targets as reference antibodies, with this method having to be sufficiently easy to implement to enable the screening of a large library of antibodies of known protein sequence.

### SUMMARY OF THE INVENTION

The inventors have developed a method making it possible to effectively identify, from a data bank of antibodies of known protein sequence, one or more antibodies which have the same antigenic specificity as a reference antibody.

The present invention relates to a method making it possible to identify antibodies capable of binding to the same target, the method comprising:
providing a database comprising the characterization of at least 100 antibodies, each antibody being characterized by a simplified sequence and a secondary structure of the CDRs (complementarity-determining regions) CDR1, CDR2 and CDR3 for each variable domain of an antibody of the database, and
   the simplified sequence being a translated sequence in which each amino acid of the initial sequence of the CDRs is replaced by a code representing a category of amino acids, the categories being based on the physicochemical characteristics of the amino acids, and the number of categories being between 4 and 10; and
   the secondary structure being a translated sequence in which each amino acid of the initial sequence of the CDRs is replaced by a code representing a type of secondary structure, the number of types of secondary structure being between 3 and 8;
providing a simplified sequence and a secondary structure of the CDRs CDR1, CDR2 and CDR3 for a variable domain of a reference antibody;
calculating a score for similarity between the variable domain of the reference antibody and a variable domain of a test antibody from the database based on the simplified sequence of their CDRs and on the secondary structure of their CDRs; and
selecting the test antibody if the similarity score is such that it predicts that the test antibody and the reference antibody are capable of binding to the same target, the test antibody selected being referred to as similar antibody.

In a preferred embodiment, the categories based on the physicochemical characteristics of the amino acids are chosen from the size of the amino acids, their hydrophobicity, their polarity, their charge, their aromatic nature and combinations of these characteristics. For example, the categories chosen are:
small size for the amino acids A, G, S, T, C and P;
aromatic nature for the amino acids Y, F and W;
hydrophobicity for the amino acids I, L, F, M and V;
polarity for the amino acids N and Q;
positive charge for the amino acids H, K and R; and
negative charge for the amino acids D and E.

In a preferred embodiment, the types of secondary structure are chosen from the following, or are the following:
3₁₀ helix;
α helix;
π helix;
tight turn;
β strand;
residue in a β-bridge;
turn; and
absence of secondary structure.

Preferably, the similarity score is calculated by identifying subsequences common to the CDRs of the reference antibody and of the test antibody.

In a preferred embodiment, the method also comprises a step of in vitro validation of the binding capacity of the similar antibody to the target of the reference antibody or of the reference antibody to the target of the similar antibody.

In a preferred embodiment, the method comprises a step of in vitro validation of the binding capacity of the similar antibody to the target of the reference antibody. This validation step may comprise providing or producing the similar antibody, bringing it into contact with the target of the reference antibody, and measuring the binding between the similar antibody and the target of the reference antibody. Preferably, the CDR1, CDR2 and CDR3 are grafted into a nano-antibody backbone. The method may comprise a step of selecting the similar antibody if the binding capacity of the similar antibody to the target of the reference antibody is validated in vitro.

In a preferred embodiment, the method comprises a step of in vitro validation of the binding capacity of the reference antibody to the target of the similar antibody. This validation step may comprise producing or providing the reference antibody, bringing it into contact with the target of the similar antibody, and measuring the binding between the reference antibody and the target of the similar antibody. This validation step may comprise a step of selecting the reference antibody if the binding capacity of the reference antibody to the target of the similar antibody is validated in vitro.

In a particular embodiment, the antibodies are nano-antibodies.

### DESCRIPTION OF THE FIGURES

FIG. 1. General diagram of the method. A: At input, there is an initial antibody or a reference antibody (initial Nb). If this is a bivalent antibody, the variable domains of the heavy chain (VH) and of the light chain (VL) are considered as nano-antibodies (Nb) and treated separately. B: Either the 3D structure is available in the Protein Data Bank (PDB) or a model is produced by homology. C: extraction of the CDRs. D: recoding by simplification of the protein sequence and annotation of the secondary structures. E: calculating the similarity of the initial Nb with all the Nbs of the database. F: list of candidates ordered by the similarity score. G: the database used consists of sequences of Nb, corresponding to monovalent antibodies or to VHs or VLs of bivalent antibodies, the 3D structure of which is either known or modeled by homology, and which are recoded in the same way as the initial Nb.

FIG. 2. Principle of recoding of the CDR sequences. Simplification and annotation of the Nb sequences. Simplified and annotated sequences of the CDRs of the Nbs considered in the examples.

FIG. 3. Effects of the antibodies on the recruitment of β-arrestins. HEK293 cells were transiently transfected with CXCR4 fused to RLuc8 and β-arrestin 2 fused to YFP. Forty-eight hours after transfection, the cells are exposed to different VHHs at the indicated concentrations for 2 hours at 37° C. in 10 mM PBS-Hepes. They were subsequently stimulated with SDF1 at 50 nM. The BRET signal is read 15 minutes after the stimulation and after addition of Coelenterazine H at 5 μM. The results are expressed as mean±SEM of n>3 independent experiments carried out in triplicate.

FIG. 4. Inhibition of the Gi pathway by the antibodies. HEK293 cells were transiently transfected with CXCR4 and the cAMP sensor CAMYEL. Forty-eight hours after transfection, the cells are exposed to different VHHs at the indicated concentrations for 2 hours at 37° C. in 10 mM PBS-Hepes. They were subsequently stimulated with SDF1 at 50 nM. The BRET signal is read 15 minutes after the stimulation and after concomitant addition of Coelenterazine H at 5 μM and forskolin at 100 μM. The results are expressed as mean±SEM of n>3 independent experiments carried out in triplicate.

FIG. 5. Binding of 3SM5, 238D2, 238D4 and 4N1H to hemagglutinin (H1N1), target of 3SM5. The surfaces of ELISA plates are loaded with hemagglutinin H1N1 at 2 μg/ml and overloaded with PBS-1% milk. The VHHs were incubated for 2 hours at room temperature at 1 μM of PBS and the secondary antibody anti-c-myc-HRP was incubated at 1:1000 for 1 h at room temperature. Revealing is carried out using ECL. The results are expressed as mean±SEM of the percentage of the maximum response obtained in each of the 5 experiments carried out in triplicate.

FIG. 6. 3sm5 binds CXCR4, like 238D2 and 238D4. HEK293 cells were transiently transfected with CXCR4. Forty-eight hours after the transfection, the cells are exposed to the VHHs 3SM5 (A), 238D2 (B), 238D4 (C), and ovalbumin (A, B, C) at 1 μM for 1 hour at room temperature. The binding of the VHHs to the cells is revealed using an anti-His antibody coupled to allophycocyanin (APC). The fluorescence is measured using FACSCalibur.

FIG. 7. 3SM5 and 4N1H bind CXCR4, like 238D2. HEK293 cells were transiently transfected with CXCR4. Forty-eight hours after the transfection, the cells are exposed to the nano-antibodies 3SM5 (A), 4N1H (B), 238D2 (C) and ovalbumin (A, B, C) at 100 nM for 1 hour at 4° C. The binding of the nano-antibodies is revealed using an anti-His antibody coupled to allophycocyanin (anti-His-APC). The measurement is carried out using the MACSQuant Analyzer 10 (Miltenyi Biotec).

FIG. 8. 3SM5 and 4N1H bind the same epitope on CXCR4 as 238D2. Peptides corresponding to the indicated segments of CXCR4 were synthesized and N-terminally bonded to a biotin. Each peptide (at 2 mM) was brought into the presence of 2 μM of nano-antibodies coupled to the His tag, with 50 nM of anti-His antibody coupled to an energy donor, terbium, and with 2.35 μM of streptavidin coupled to the energy acceptor d2. The energy transfer between Tb and d2 is measured using the Mithras² LB 943 Monochromator Multimode Reader (Berthold Technologies).

FIG. 9. 3SM5 and 4N1H displace the ligand SDF1 of CXCR4, like 238D2. HEK293 cells were transiently transfected with CXCR4 fused to the SNAP tag (Cisbio Bioassays). Forty-eight hours after the transfection, the cells are treated to insert the terbium donor onto the SNAP tag. The ligand SDF1 coupled to the acceptor d2 is added at 12 nM. The cells are subsequently incubated with the nano-antibodies 3SM5, 4N1H, 238D2 and ovalbumin at the indicated concentrations for 1 hour. The energy transfer is measured using the Mithras² LB 943 Monochromator Multimode Reader (Berthold Technologies).

FIG. 10. 238D2 immunoprecipitates with H1N1, like 3SM5. Magnetic beads were coupled with an anti-myc antibody and exposed to a mixture of H1N1 and nano-antibodies coupled to the myc tag and to the biotinylated AviTag for 1 hour at 4° C. The beads were washed several times and eluted. The eluates were deposited in acrylamide gel then transferred to nitrocellulose membrane. The membranes were hybridized with a streptavidin coupled to AlexaFluor680 to reveal the nano-antibodies (bottom box) and to an anti-His antibody to reveal H1N1 (top box). Aliquots of the purified proteins that had not undergone the immunoprecipitation process were also deposited in the gel and appear under the name “input”.

FIG. 11. 238D2 and 3SM5 bind H1N1 with the same affinity. Interferometry streptavidin biosensors are loaded with the nano-antibodies coupled to the biotinylated AviTag, then exposed to H1N1 at different concentrations ranging from 84.75 to 847.5 nM. Dissociation takes place in the working buffer. The dissociation constants (Kd) indicated on the graphs were obtained by the simultaneous mathematical fitting of all the curves.

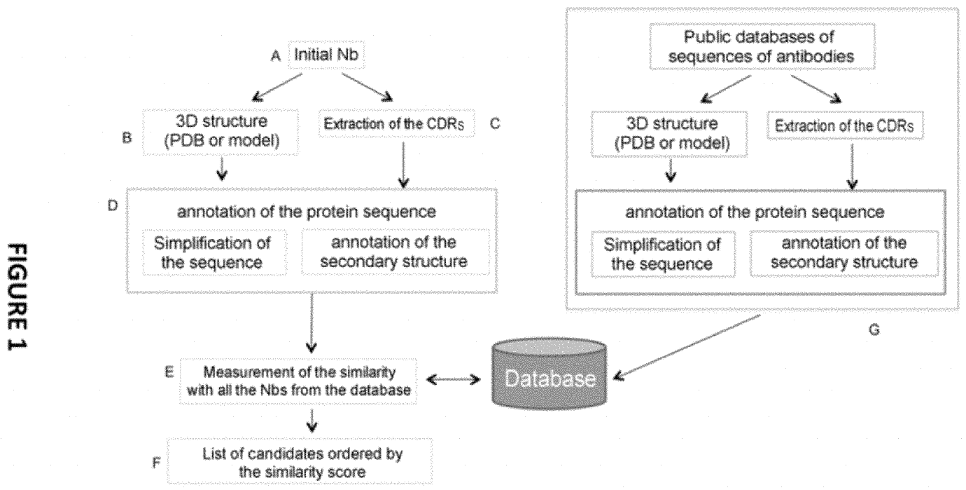

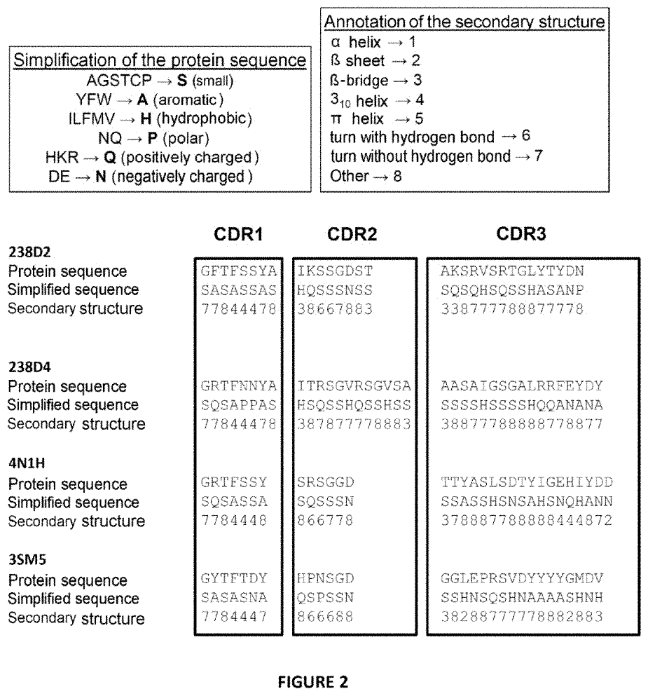

### DETAILED DESCRIPTION OF THE INVENTION

The present invention relates to a method which makes it possible, from a reference antibody, to identify antibodies of the same specificity (that is to say binding to the target of the reference antibody) from a data bank comprising antibody characterizations. Similarly, the present invention also relates to a method which makes it possible, from a reference antibody, to determine other targets of this antibody.

Thus, the method according to the present invention is suitable:
for identifying new antibodies specific to a given target based on a reference antibody specific to said target; or
for identifying new targets for a given reference antibody.

In the present application, “target” of an antibody denotes a biological molecule which is specifically bound by the antibody. The term “target” may also be replaced by “antigen”.

This method is based on measuring the similarity of the reference antibody to antibodies contained in a library. Thus, the database comprises the characterization of at least 100, 150, 200 or 1000 antibodies.

In this database, the antibodies are characterized by the properties of their CDRs (complementarity-determining region).

In particular, the method is based on an analysis of the CDRs, making it possible to quantify similarities between a model or reference antibody and antibodies of the database, especially by counting the subsequences common to the CDRs.

Preferably, the step of in silico prediction is followed by a step of in vitro experimental validation. This step of experimental validation may optionally comprise in vivo tests in animal models.

The method developed by the inventors was validated, as shown in the examples, by applying the method of the present invention for identifying antibodies capable of targeting the receptor CXCR4. In particular, based on two antibodies known to bind specifically to the receptor CXCR4, the inventors succeeded in identifying two other antibodies capable of binding to CXCR4, even though their known targets had no relationship to CXCR4. Indeed, these two antibodies have an antagonistic effect on the activity of the receptor, just like the reference antibody used to identify them. Finally, the inventors also observed that the reference antibody used to identify the two new antibodies is capable of binding to the target of one of the new antibodies. Moreover, it is interesting to note that these antibodies could not have been identified by conventional methods.

Thus, the method according to the present invention is suitable for the identification, from a reference antibody, of other antibodies having the same specificity and similar pharmacological properties to those of the reference antibody. Thus, when an antibody has potentially beneficial pharmacological properties but physicochemical properties which make it unusable (for example toxicity, low solubility, difficulty of production), this method may make it possible to find a new candidate having the same potential without the associated drawbacks.

The method also makes it possible, for a given antibody, to determine possible targets with which this antibody is liable to interact. In this context, this method may make it possible to anticipate potential cross-reactions, possible sources of harmful side effects, upstream in the development of a therapeutic antibody.

This method therefore makes it possible to very quickly identify antibodies having the same specificity as a reference antibody and also alternative targets for this reference antibody, since the search is carried out in silico. This identification is preferably followed by a step of validation by in vitro experiments.

Database According to the Present Invention

The database according to the present invention comprises characterizations for each antibody. In particular, it comprises, for each antibody:
a simplified sequence of the CDRs, and
the secondary structures of the CDRs.

In particular, the database according to the present invention comprises, for each variable domain:
a complete sequence of the variable domain;
a simplified sequence of the CDRs of the variable domain;
a secondary structure of the CDRs of the variable domain; and
if available, the target recognized by the antibody comprising the variable domain and in particular the epitope recognized by the antibody.

Thus, as detailed below, each amino acid of the sequences of the CDRs is assigned a set of attributes, these attributes being defined by the biochemical nature of the amino acid and by the type of secondary structure to which it contributes.

The database is constructed based on the sequences of the variable domains of the antibodies.

The data of the antibodies introduced into the database correspond to nano-antibodies (nanobody).

Nano-antibodies or single-domain antibodies are well-known natural antibodies consisting of heavy-chain antibodies lacking light chains. The variable domain thereof is typically formed of a plurality of regions, including a plurality of conserved framework regions, referred to as FR, and a plurality of hypervariable regions that determine complementarity with the antigen, referred to as CDRs.

Thus, if the initial antibody is a bivalent antibody such as an immunoglobulin (IgG), the variable domains of the heavy chain and of the light chain are each considered to be nano-antibodies and are treated separately. If the initial antibody is a single-chain antibody (scFv), the two variable parts are considered, and as with an IgG, the variable domain of the heavy chain and that of the light chain are each considered to be nano-antibodies and are treated separately.

The sequence of the variable domain is translated into a simplified code. Thus, each amino acid is replaced by a code representing a category of amino acids, the categories being based on the physicochemical characteristics of the amino acids (for example Bourquard et al., PLoS One, 2011, 6(4): e18541). Optionally, the simplified sequence may be solely determined for the sequences of the CDRs of the variable domains and not over the whole sequence of the variable domain.

The number of categories used to recode the sequences is less than 15, preferably than 10. The number of categories may be between 4 and 10, preferably between 5 and 9, even more preferably is approximately 6.

The categories based on the physicochemical characteristics of the amino acids are or are chosen from the size of the amino acids, their hydrophobicity, their neutrality, their polarity, their charge, their aromatic nature and combinations of these characteristics. The categories may thus be one or more categories chosen from the following: very small, small, aliphatic, aromatic, hydrophobic, polar, positive charge, negative charge, and combinations thereof.

For example, in a particular embodiment, the categories used are the following:
“small”, comprising the following amino acids, A, G, S, T, C and P and denoted by S;
“aromatic”, comprising the following amino acids: Y, F and W and denoted by A;
“hydrophobic”, comprising the following amino acids: I, L, F, M and V and denoted by H;
“polar”, comprising the following amino acids: N and Q and denoted by P;
“positive charge”, comprising the following amino acids: H, K and R and denoted by Q;
“negative charge”, comprising the following amino acids: D and E and denoted by N.

Examples of simplification of sequences are illustrated in the present application.

The database also comprises the secondary structure of the CDRs.

The secondary (2D) structure may be determined by any appropriate software. Any method making it possible to determine the secondary structure may be used. There are several methods to define the secondary structure of a protein (for example, STRIDE (Frishman D., Argos P., Proteins, vol. 23, no 4, 1995, p. 566-579); or DEFINE (Richards F. M., Kundrot C. E., Proteins, vol. 3, no 2, p.p, 1988 0.71-84). One of the preferred methods is the method of the dictionary of protein secondary structure (DSSP) (Touw et al. Nucleic Acids Research 2015; 43: D364-D368. Kabsch & Sander. Biopolymers. 1983, 22, 2577-2637). The latter is commonly used to describe the structure of proteins using one-letter codes.

In particular, the secondary structure is determined by software using a three-dimensional (3D) structure of the variable domain.

The 3D structure of the variable domain may be available in a database, in particular a public database such as PDB (protein data bank). This PDB bank is a worldwide collection of data on the three-dimensional structure of biological macromolecules, including antibodies. When the information is available, the characteristics of secondary structure may also be available in the PDB database.

If the 3D structure is not available, then it can be obtained by modeling by homology, preferably taking the antibody of known structure, whose CDRs are closest to those of the antibody to be modeled (same lengths and maximized sequence identity), as the support structure. Software is available for carrying out this modeling, for example the modeler software (Webb et al, Current Protocols in Bioinformatics, John Wiley& Sons, Inc., 5.6.1-5.6.32, 2014; Marti-Renom et al. Annu. Rev. Biophys. Biomol. Struct. 29, 291-325, 2000; Sali & Blundell. J. Mol. Biol. 234, 779-815, 1993; Fiser et al. Protein Science 9. 1753-1773, 2000).

Thus, each amino acid of the CDRs is associated with a secondary structure. The categories of secondary structures may for example be chosen from the following:
3₁₀ helix, which may for example be denoted by G;
α helix, which may for example be denoted by H;
π helix, which may for example be denoted by I;
tight turn, which may for example be denoted by T;
β strand, which may for example be denoted by E;
residue in a β-bridge, which may for example be denoted by B;
turn, which may for example be denoted by 5;
absence of secondary structure.

Alternatively, the secondary structure categories may also be encoded by a number.

Thus, each residue of the CDRs may be associated with a secondary structure category, which may optionally group together several categories as described above.

Thus, the CDRs may also be translated into a sequence in which each amino acid of the initial sequence of the CDRs is replaced with a code representing a secondary structure category and the number of categories being between 3 and 10, preferably between 4 and 9, even more preferably between 5 and 8.

The CDRs of the variable domains may be extracted by any method available to those skilled in the art. In a preferred embodiment, the method of Chothia is used for determining the CDRs (Chothia et al, Nature 342, 877-883). However, any other alternative method for determining CDRs may be used as long as the method used for the database is homogeneous therein. For example, and non-limitingly, mention may be made of the method of Kabat (Kabat et al., 1991, Sequences of Proteins of Immunological Interest, 5th Ed., United States Public Health Service, National Institutes of Health, Bethesda), the method intermediate between Chothia and Kabat, referred to as AbM (antibody modeling software AbM from Oxford Molecular) or the “contact” method based on an analysis of available complex structures.

Finally, the database also comprises, when the information is available, the target of the antibody, and in particular optionally the epitope bound by the antibody.

The present invention therefore also relates to such a data bank comprising a plurality of variable domains, with, for each variable domain, a simplified sequence of the CDRs of each variable domain and the secondary structure of the CDRs of each variable domain. Preferably, the database also comprises the target or targets of the antibody comprising the variable domain. The database may comprise additional information for each variable domain, such as the sequence of the epitope recognized by the antibody, the biological activity of the antibody on the target (for example agonistic, antagonistic, blocking, inhibitory, activatory, etc.), the three-dimensional structure of the antibody, the simplified sequence of the variable domain, the model of the three-dimensional structure if it was necessary to produce it, and in this case the support structure used for the modeling.

Preferably, the database comprises at least 100, 150, 200, 250 or 300 antibodies. In a preferred embodiment, the database comprises at least 2 different antibodies per target, preferably at least 3 antibodies. In a preferred embodiment, the database comprises all the antibodies for which the protein sequence is known and for which either the three-dimensional structure is available or a model thereof by homology was able to be produced.

Similarity

Based on the simplified sequences of the CDRs and the secondary structures of the CDRs of the data bank, the method comprises a calculation of similarity between a reference antibody and antibodies of the database according to the present invention.

There are a large number of available methods for calculating similarities.

In a preferred embodiment, the method is based on counting subsequences common to the CDRs of two antibodies, thereby making it possible to quantify the similarity of the reference antibody and of another test antibody of the database. This similarity is measured by a score which corresponds to a distance between two antibodies and is based on the counting of subsequences. The similarity calculation method was developed based on the teaching of Egho et al (Research Report, RR-8086, 2012, 1-19).

A short distance (that is to say a large degree of similarity) is indicative of a capacity to bind to the same target as the reference antibody. An additional item to take into account is the number of reference antibodies having the same target and which have a high similarity with the same test antibody. If several reference antibodies have a high degree of similarity, then the distance value may be a bit higher than when a single reference antibody has similarity.

Reference or threshold numerical values of similarity may be readily defined by the user in order to define the reference or threshold numerical value of similarity of use for predicting if the test antibody and the reference antibody are capable of binding to the same target. For example, these values could be determined with antibodies known to bind the same target and optionally with antibodies known not to bind to the same targets.

Thus, those antibodies at a distance with a value of less than or equal to 4 relative to a reference antibody are predicted to be able to bind to the same target as the reference antibody.

Additionally, those antibodies at a distance with a value of between 4 and 6 relative to several reference antibodies having the same target are predicted to be able to bind to the same target as the reference antibodies.

The antibodies corresponding to a value predicting target similarity are selected. These selected antibodies are denoted by the name similar antibody. These antibodies may subsequently be subjected to experimental validation in order to test their specificity for the target.

Experimental Validation

Preferably, the in silico identification of a potential antibody capable of binding to the target, or similar antibody, is validated experimentally. Thus, this validation step may make it possible to confirm the capacity of a similar antibody to bind to the target of the reference antibody or to confirm the capacity of a reference antibody to bind to the target of the similar antibody.

The method according to the present invention may thus comprise an additional step of in vitro validation of the binding capacity of the similar antibody or the reference antibody to the target of the reference antibody or the target of the similar antibody, respectively.

Thus, in a first embodiment, the validation step is a step of in vitro validation of the binding capacity of the similar antibody to the target of the reference antibody.

The method may comprise providing or producing the identified antibody or similar antibody, bringing it into contact with the target of the reference antibody, and measuring the binding between the identified or similar antibody and the target.

Preferably, the identified antibody or similar antibody may be provided in its original form or the CDRs (in particular CDR1, CDR2 and CDR3) of this antibody may be grafted into a suitable backbone. In a preferred embodiment, the preferred backbone will be that of a nano-antibody, also referred to as VHH. Alternatively, the backbone into which the CDRs may be grafted is that of the heavy chain VH of an antibody. In this context, the heavy chain will or will not be associated with a light chain.

In a second embodiment, the validation step is a step of in vitro validation of the binding capacity of the reference antibody to the target of the similar antibody.

The method may comprise producing or providing the reference antibody, bringing it into contact with the target of the similar antibody, and measuring the binding between the reference antibody and the target of the similar antibody.

The reference antibody may be provided in its original form or the CDRs (in particular CDR1, CDR2 and CDR3) of this antibody may be grafted into a suitable backbone. In a preferred embodiment, the preferred backbone will be that of a nano-antibody, also referred to as VHH. Alternatively, the backbone into which the CDRs may be grafted is that of the heavy chain VH of an antibody. In this context, the heavy chain will or will not be associated with a light chain.

In a preferred embodiment, the backbone of a nano-antibody, also referred to as VHH, will be preferred for the step of in vitro validation. Thus, the CDRs (CDR1, CDR2 and CDR3) are grafted into a nano-antibody backbone comprising framework regions FR1, FR2, FR3 and FR4 in order to form a nano-antibody comprising the segments FR1-CDR1-FR2-CDR2-FR3-CDR3-FR4 (a first framework region FR1, a first hypervariable region CDR1, a second framework region FR2, a second hypervariable region CDR2, a third framework region FR3, a third hypervariable region CDR3, and a fourth framework region FR4).

FR1, FR2, FR3 and FR4 may exist in VHH regions of Camelidae, preferably of dromedary, camel, llama or alpaca. FR1, FR2, FR3 and FR4 may also be humanized VHH regions. For example, application WO2015/063331 illustrates the preparation of synthetic single-domain antibodies.

Numerous nano-antibody backbones are now available. For example, the first framework region could be chosen from MEVQLQESGGGLVQAGASLKLSCAAS (SEQ ID No 1) or MEVQLVQSGAEVKKPGASVKVSCKAS (SEQ ID No 2), the second framework region could be chosen from MGWFRQAPGKEREFVAA (SEQ ID No 3) or HINWVRQAPGQGLEWMGW (SEQ ID No 4), the third framework region could be chosen from TKYADSVKGRFAISRDNDKNTVWLRMNSLKPEDTAVYYC (SEQ ID No 5) or NYAQKFQGWVTMTRDTAISTAYMEVNGLKSDDTAVYYC (SEQ ID No 6), and the fourth framework region could be WGQGTQVTVSS (SEQ ID No 7), or any sequences having at least 80, 85, 90 or 95% identity with these. In a particular embodiment, the first framework region could be MEVQLQESGGGLVQAGASLKLSCAAS (SEQ ID No 1), the second framework region could be MGWFRQAPGKEREFVAA (SEQ ID No 3), the third framework region could be TKYADSVKGRFAISRDNDKNTVWLRMNSLKPEDTAVYYC (SEQ ID No 5), and the fourth framework region could be WGQGTQVTVSS (SEQ ID No 7), or any sequences having at least 80, 85, 90 or 95% identity with these. In a second particular embodiment, the first framework region could be MEVQLVQSGAEVKKPGASVKVSCKAS (SEQ ID No 2), the second framework region could be HINWVRQAPGQGLEWMGW (SEQ ID No 4), the third framework region could be NYAQKFQGWVTMTRDTAISTAYMEVNGLKSDDTAVYYC (SEQ ID No 6), and the fourth framework region could be WGQGTQVTVSS (SEQ ID No 7), or any sequences having at least 80, 85, 90 or 95% identity with these.

This step of validation may be carried out by any method available to those skilled in the art. Non-exhaustively, mention may for example be made of ELISA assays, interferometry or surface plasmon resonance assays, or any other method involving the transfer of energy between fluorescent molecules.

The implementation of the method is illustrated in the following examples.

### EXAMPLES

### Example 1—Identification of New Antibodies Targeting the Receptor CXCR4 and Identification of an Alternative Target for the Reference Antibody

Summary

The proof of concept of the method was carried out using an antibody targeting the receptor CXCR4 (238D2) as reference antibody. This antibody was described in the publication by Jähnichen et al. (2010, PNAS, 107, 20565-70; US 2011/0117113 A1).

Two homologous antibodies were identified by the method according to the present invention: a camelid VHH targeting β-lactamase (PDB code 4N1H) and a VH domain of a human antibody targeting the hemagglutinin of the H1N1 virus (strain A/Solomon Islands/3/2006, PDB code 3SM5).

Neither of these two antibodies had been tested for its capacity to recognize the receptor CXCR4, and there is no overall resemblance either in terms of sequence or in terms of structure between CXCR4 and β-lactamase or between CXCR4 and hemagglutinin.

Using cytometry experiments, the inventors showed that 3SM5 and 4N1H recognize the human CXCR4 receptor expressed transiently in HEK293 cells. FRET and BRET studies with these same cells made it possible to show that the two antibodies 3SM5 and 4N1H inhibit the activity of the receptor CXCR4, as does the reference antibody.

The inventors were able to demonstrate experimentally that, like the reference antibody, the two antibodies identified in silico by the method according to the invention inhibit the activation of the G-protein dependent signaling pathway and also the recruitment of β-arrestins by the receptor CXCR4 activated by its natural ligand, the chemokine SDF-1α. The antagonistic activity of the identified antibodies occurs from concentrations of the order of one nM and is dependent on the dose of the antibody. A VHH directed against ovalbumin was used as negative control; while it has low activity at doses of the order of one nM, this activity is not dose-dependent and is therefore non-specific.

Results

The method was applied taking, as initial antibodies, the variable domains of the VHHs “238D2” and “238D4” which recognize the human CXCR4 receptor.

Since the 3D structures are not available, 3D models were produced using the PDB structure 3SN6 as support for the two antibodies.

The inventors were thus able to identify two candidate antibodies:
The VH domain of the antibody CH65, which targets the hemagglutinin of the H1N1 strain of the influenza virus (strain A/Solomon Islands/3/2006), PDB code 3SM5 (Whittle, et al. (2011). Proceedings of the National Academy of Sciences, 108(34), 14216-14221). This nano-antibody has a similarity of 3.6 with the initial nano-antibody 238D2.
The variable domain of the single-chain antibody cAB-F11N, which targets the R-lactamase of the bacterium Bacillus licheniformis, PDB code 4N1H (not published). This nano-antibody has a similarity of 4.2 with the nano-antibody 238D4 and of 6 with the nano-antibody 238D2.

The variable domains of cAB-F11N (hereinafter referred to as 4N1H) and of the heavy chain of CH65 (hereinafter referred to as 3SM5) were produced using the sequence reported in the PDB, adding a myc tag and a his tag.

4N1H:
SEQ ID NO 8:
MEVQLQESGGGLVQAGASLKLSCAASGRTFSSYAMGWFRQAPGKEREFV
AAISRSGGDTKYADSVKGRFAISRDNDKNTVWLRMNSLKPEDTAVYYCA
ATTYASLSDTYIGEHIYDDWGQGTQVTVSS
SEQ ID NO 9 (4N1H-myc tag):
MEVQLQESGGGLVQAGASLKLSCAASGRTFSSYAMGWFRQAPGKEREFV
AAISRSGGDTKYADSVKGRFAISRDNDKNTVWLRMNSLKPEDTAVYYCA
ATTYASLSDTYIGEHIYDDWGQGTQVTVSSEQKLISEEDLE
SEQ ID NO 10 (4N1H-his tag):
MEVQLQESGGGLVQAGASLKLSCAASGRTFSSYAMGWFRQAPGKEREFV
AAISRSGGDTKYADSVKGRFAISRDNDKNTVWLRMNSLKPEDTAVYYCA
ATTYASLSDTYIGEHIYDDWGQGTQVTVSSEQKLISEEDLEHHHHHH
3SM5
SEQ ID NO 11:
MEVQLVQSGAEVKKPGASVKVSCKASGYTFTDYHINWVRQAPGQGLEWM
GWIHPNSGDTNYAQKFQGWVTMTRDTAISTAYMEVNGLKSDDTAVYYCA
RGGLEPRSVDYYYYGMDVWGQGTTVTVSS
SEQ ID NO 12 (3SM5-myc tag):
MEVQLVQSGAEVKKPGASVKVSCKASGYTFTDYHINWVRQAPGQGLEWM
GWIHPNSGDTNYAQKFQGWVTMTRDTAISTAYMEVNGLKSDDTAVYYCA
RGGLEPRSVDYYYYGMDVWGQGTTVTVSSEQKLISEEDLE
SEQ ID NO 13 (3SM5-his tag):
MEVQLVQSGAEVKKPGASVKVSCKASGYTFTDYHINWVRQAPGQGLEWM
GWIHPNSGDTNYAQKFQGWVTMTRDTAISTAYMEVNGLKSDDTAVYYCA
RGGLEPRSVDYYYYGMDVWGQGTTVTVSSEQKLISEEDLEHHHHHH
SEQ ID NO 38 (AviTag-3SM5-myc tag)
MGSSHHHHHHSSGLVPRGSHMSGLNDIFEAQKIEWHEDPNSEVQLVQSG
AEVKKPGASVKVSCKASGYTFTDYHINWVRQAPGQGLEWMGWIHPNSGD
TNYAQKFQGWVTMTRDTAISTAYMEVNGLKSDDTAVYYCARGGLEPRSV
DYYYYGMDVWGQGTTVTVSSEQKLISEEDL
238D2
SEQ ID NO 39 (238D2-myc tag)
MEVQLVESGGGLVQTGGSLRLSCAASGFTFSSYAMSWVRQAPGKGLEWV
SGIKSSGDSTRYAGSVKGRFTISRDNAKNMLYLQMYSLKPEDTAVYYCA
KSRVSRTGLYTYDNRGQGTQVTVSSEQKLISEEDLE
SEQ ID NO 40 (238D2-his tag)
MEVQLVESGGGLVQTGGSLRLSCAASGFTFSSYAMSWVRQAPGKGLEWV
SGIKSSGDSTRYAGSVKGRFTISRDNAKNMLYLQMYSLKPEDTAVYYCA
KSRVSRTGLYTYDNRGQGTQVTVSSEQKLISEEDLEHHHHHH
SEQ ID NO 41 (AviTag-238D2-myc tag)
MGSSHHHHHHSSGLVPRGSHMSGLNDIFEAQKIEWHEDPNSEVQLVESG
GGLVQTGGSLRLSCAASGFTFSSYAMSWVRQAPGKGLEWVSGIKSSGDS
TRYAGSVKGRFTISRDNAKNMLYLQMYSLKPEDTAVYYCAKSRVSRTGL
YTYDNRGQGTQVTVSSEQKLISEEDL
238D4
SEQ ID NO 42 (238D4-myc tag)
MEVQLMESGGGLVQAGGSLRLSCAASGRTFNNYAMGWFRRAPGKEREFV
AAITRSGVRSGVSAIYGDSVKDRFTISRDNAKNTLYLQMNSLKPEDTAV
YTCAASAIGSGALRRFEYDYSGQGTQVTVSSEQKLISEEDLE
SEQ ID NO 43 (238D4-his tag)
MEVQLMESGGGLVQAGGSLRLSCAASGRTFNNYAMGWFRRAPGKEREFV
AAITRSGVRSGVSAIYGDSVKDRFTISRDNAKNTLYLQMNSLKPEDTAV
YTCAASAIGSGALRRFEYDYSGQGTQVTVSSEQKLISEEDLEHHHHHH
Ovalbumin
SEQ ID NO 44 (Ovalbumin-his tag)
MGSSHHHHHHSSGLVPRGSHMASMTGGQQMGRGSEFELRRQACGRTRAP
PPPPLRSGCEVQLQESGGSGQAGGSLRLSCAASGDTVRTMAWFRQAPGQ
EREGVAGFNLPISRPYYADGMKARFTISGDKSKNTVTLQMDNLAPEDTA
NYYCAATRYTLDLSSRIFQGDFDHWGHGTQVTVSS
SEQ ID NO 45 (CXCR4)
MEGISIYTSDNYTEEMGSGDYDSMKEPCFREENANFNKIFLPTIYSIIF
LTGIVGNGLVILVMGYQKKLRSMTDKYRLHLSVADLLFVITLPFWAVDA
VANWYFGNFLCKAVHVIYTVNLYSSVWILAFISLDRYLAIVHATNSQRP
RKLLAEKVVYVGVWIPALLLTIPDFIFANVSEADDRYICDRFYPNDLWV
VVFQFQHIMVGLILPGIVILSCYCIIISKLSHSGSNIFEMLRIDEGLRL
KIYKDTEGYYTIGIGHLLTKSPSLNAAKSELDKAIGRNTNGVITKDEAE
KLFNQDVDAAVRGILRNAKLKPVYDSLDAVRRAALINMVFQMGETGVAG
FTNSLRMLQQKRWDEAAVNLAKSRWYNQTPNRAKRVITTFRTGTWDAYG
SKGHQKRKALKTTVILILAFFACWLPYYIGISIDSFILLEIIKQGCEFE
NTVHKWISITEALAFFHCCLNPILYAFLGAKFKTSAQHALTSVSRGSSG
RPLEVLFQ

The antibodies were produced in E. coli.

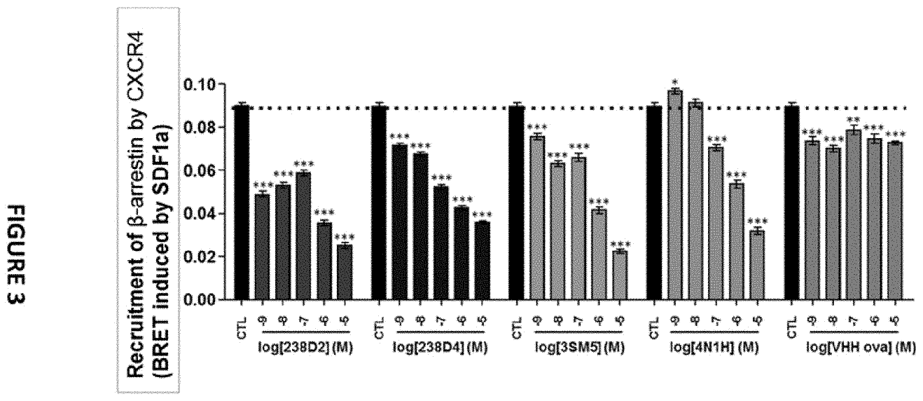

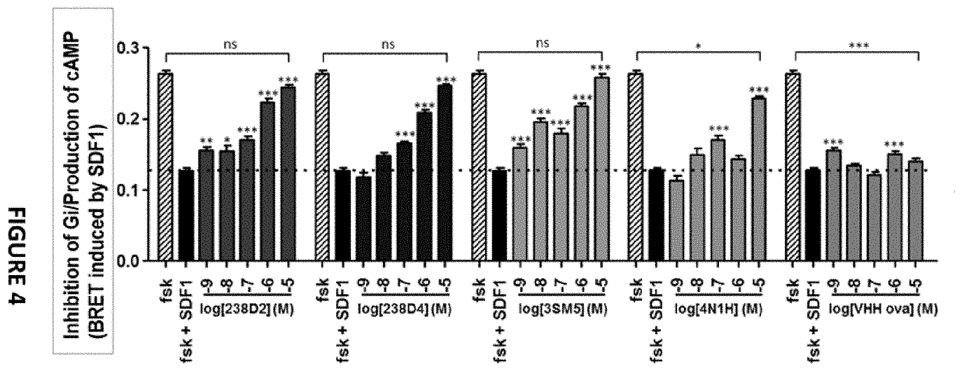

Different functional assays were subsequently carried out to demonstrate the functional effect and the specificity of the two new antibodies identified.
The ligand-dependent effect on the recruitment of β-arrestins (FIG. 3). Cells expressing the receptor are exposed to a fixed dose of SDF-1, natural agonist of the receptor CXCR4, and at increasing doses of antibody. Recruitment of the β-arrestins is measured by the BRET signal between the C-terminal tail of the receptor and the β-arrestin. A dose-dependent inhibition of the recruitment of β-arrestins by the two new antibodies was observed, which inhibition was similar to that observed with the two initial antibodies, with comparable IC50s. A VHH targeting ovalbumin is used as negative control. No effect of this antibody is observed on the effect of the SDF-1 on its receptor.
The effect on the production of second messenger (FIG. 4). Cells expressing the receptor are exposed to a fixed dose of forskolin which has the effect of greatly increasing the amount of cAMP in the cell, then a fixed dose of SDF-1 which, by activating the Gi protein, greatly reduces this amount of cAMP, and at increasing doses of antibody. The amount of cAMP in the cell is measured by the use of the sensor CAMYEL. The inhibition in the reduction of the amount of cAMP in the cell by the four antibodies, which demonstrates the inhibition of the receptor, was observed. The IC50s calculated are comparable for the four antibodies. No effect is observed with the ovalbumin VHH.

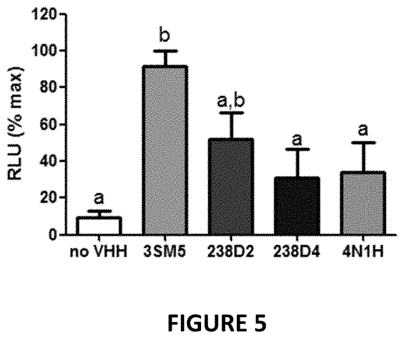

The inventors were also able to show, by ELISA, the binding of the antibodies 238D2, 238D4 and 4N1H to hemagglutinin (H1N1), the target of the antibody newly identified as being specific to CXCR4, namely 3SM5 (FIG. 5).

As shown in FIGS. 6 and 7, 3SM5 and 4N1H bind the CXCR4 overexpressed at the surface of HEK293, like 238D2 and 238D4. No binding (or very weak binding) is observed with the ovalbumin VHH.

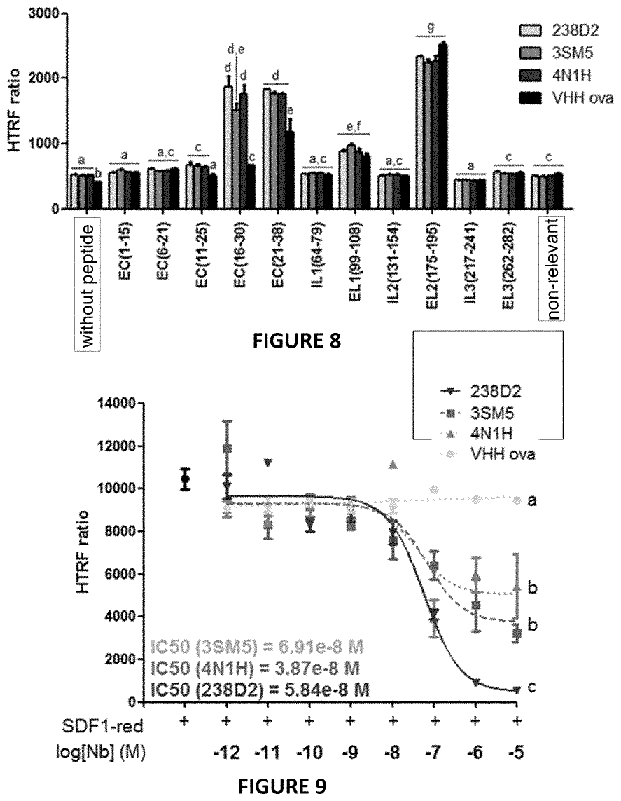

Characterization of the epitope (FIG. 8): Peptides corresponding to the segments of CXCR4 and a non-relevant peptide were synthesized and N-terminally coupled to a biotin. These peptides correspond to the following segments, the sequence of CXCR4 being described in SEQ ID NO 45 (Uniprot ID P610734):
in the extracellular domain of the receptor (EC): aa 1-15, aa 6-21, aa 11-25, aa 16-30, aa 21-38
intracellular loop 1 (IL1): aa 64-79
extracellular loop 1 (EL1): aa 99-108
intracellular loop 2 (IL2): aa 131-154
extracellular loop 2 (EL2): aa 175-195
intracellular loop 3 (IL3): aa 217-241
extracellular loop 3 (EL3): aa 262-282

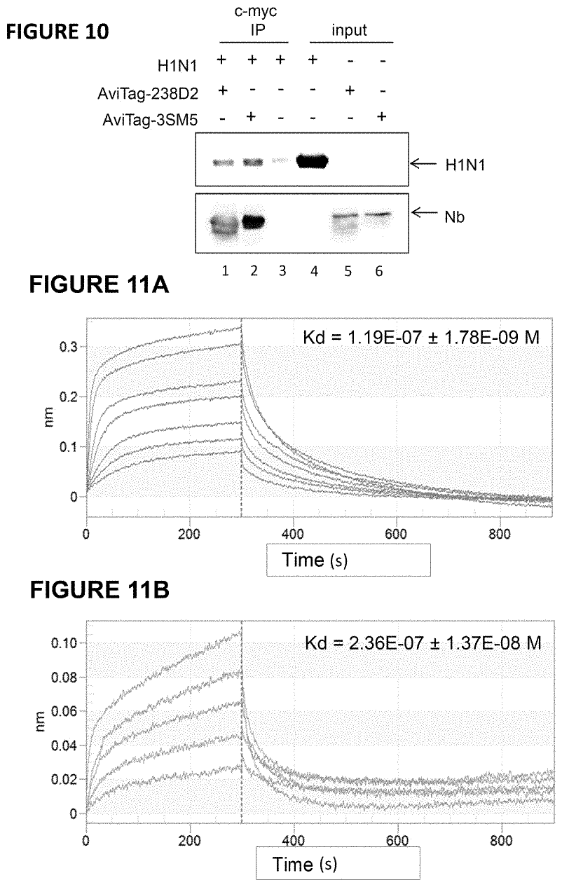

These peptides were brought into the presence of the nano-antibodies 238D2, 3SM5, 4N1H and ovalbumin, all coupled to a His tag. The binding between the peptides and the nano-antibodies was measured by HTRF (energy transfer) between a streptavidin coupled to a donor (which binds the biotin of the peptide) and an anti-His antibody coupled to the acceptor (which binds to the nano-antibodies). It was observed that the zones EC 16-30, EC 21-38, EL1 and EL2 bind the nano-antibodies 3SM5, 4N1H and 238D2, showing that the epitopes of the three antibodies are highly similar. The ovalbumin nano-antibody also binds to the zone EL2 and to a lesser extent to the zone EC 21-38 but not to the zone EC 16-30.
The effect on the binding of the ligand SDF1 (FIG. 9): Cells expressing CXCR4 coupled to a SNAP tag, to which an energy donor (terbium ion) is bound, were exposed to SDF1 coupled to an acceptor d2. A FRET signal was observed, indicating that the SDF1 binds the CXCR4. The cells were subsequently exposed to increasing doses of the nano-antibodies 238D2, 3SM5, 4N1H and ovalbumin. A decrease in the FRET signal was observed between the SDF1 and the CXCR4, indicating that the nano-antibodies 238D2, 3SM5 and 4N1H are competing with the SDF1. The IC50s are of the same order, indicating a similar affinity between the 3 nano-antibodies. The ovalbumin nano-antibody did not displace the ligand.
Co-immunoprecipitation of H1N1 and 238D2 and 3SM5 (FIG. 10): The nano-antibodies coupled to a myc tag were mixed with H1N1. The mixture was brought into the presence of magnetic beads coupled to an anti-myc antibody. It was observed that under the conditions in which 238D2 and 3SM5 are present, H1N1 precipitates onto the beads, which is not the case when the nano-antibodies are absent. H1N1 and the nano-antibody 238D2 therefore form a complex.
Measurement of affinity between 238D2 and H1N1 or 3SM5 and H1N1 (FIG. 11): Biosensors coupled to streptavidin were loaded with the nano-antibodies 238D2 and 3SM5 coupled to the biotinylated AviTag. Different doses of H1N1 were subsequently placed in the presence of the biosensors, each one inducing an association curve. Dissociation occurred passively in the working buffer. The curves are all mathematically fitted at the same time to give the Kd value (dissociation constant) indicated in the graphs. 238D2 and 3SM5 bind H1N1 with the same affinity.

Materials and Methods

Production and Purification of the Antibodies

E. coli are transformed with pET28b+ vectors encoding VHHs. The bacteria are induced with 1 mM IPTG, 4 hours at 37° C. The bacterial lyzates are purified on Ni-NDA columns (Macherey-Nagel, Düren, Germany) then the elutions are dialyzed in 100 mM Tris pH8, 500 mM NaCl, 5% glycerol at 4° C.

BRET Assays for the Detection of cAMP Production and the Recruitment of β-Arrestin 2 to the Receptor

The receptor CXCR4 was produced from cDNA (Resource Center, University of Missouri-Rolla, USA), the constructs CXCR4-Rluc8, YPET-β arrestin 2, and CAMYEL were obtained from Dr. Mohammed Akli Ayoub. HEK293 FT cells were transfected in 96-well dishes with 50 ng of plasmid and 0.5 μl of Metafectene (Biontex Laboratories GmbH, Munich, Germany) per well, in compliance with the manufacturer's instructions. Forty-eight hours after transfection, the nano-antibodies were added to the cells in 10 mM PBS-Hepes and left for 2 h at 37° C. The SDF1-α (Peprotech, Rocky Hill, N.J., USA) was added at the final concentration of 50 nM for 15 minutes. Coelenterazine H (Interchim, Montluçon, France) at the final concentration of 50 μM was added into the medium just before the measurement, as was forskolin at the final concentration of 100 μM for the CAMYEL assay since the receptor CXCR4 is coupled to the Gi protein. The plates were read with a Mithras² LB 943 Monochromator Multimode Reader (Berthold Technologies, Thoiry, France).

H1N1 ELISA

The H1N1 protein (Sino Biological Inc, Beijing, P.R.China) was deposited on Maxisorp plates at 1 μg/ml, then left overnight at 4° C. After 3 washes in PBS 0.01%-Tween 20, the plates were saturated in PBS containing 1% milk. The nano-antibodies were added for 2 h in the PBS-1% milk at the indicated concentrations. The plates were washed 3 times for 5 minutes in PBS-Tween. The HRP-coupled antibody directed against the myc tag (Abcam, Cambridge, UK) was added at the dilution of 1:000 in the PBS-1% milk and incubated for 1 h at room temperature. The plates were washed 3 times for 5 minutes in PBS-Tween and 3 times for 5 minutes in PBS. The HRP substrate (SuperSignal™ ELISA Pico Chemiluminescent Substrate, Thermo Fischer Scientific, MA, USA) diluted to 1:20 in PBS was loaded into the plates and the measurements were carried out with the Mithras² LB 943 Monochromator Multimode Reader.

FACS

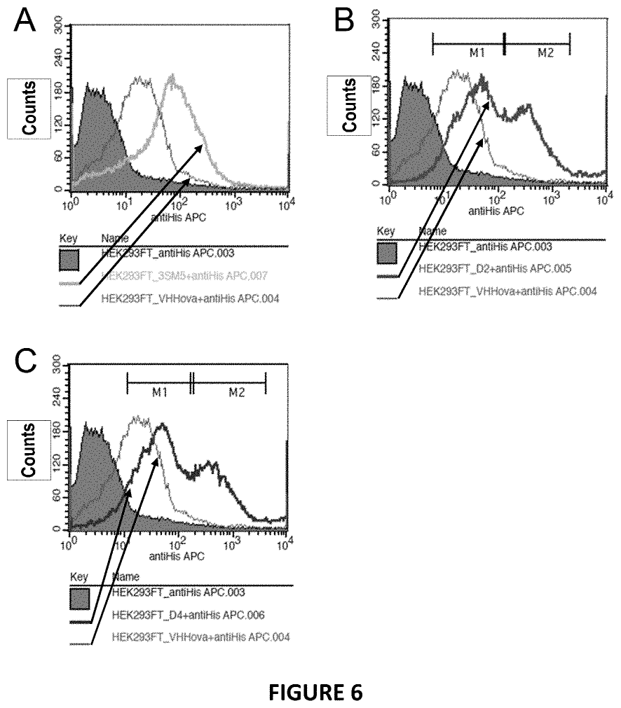

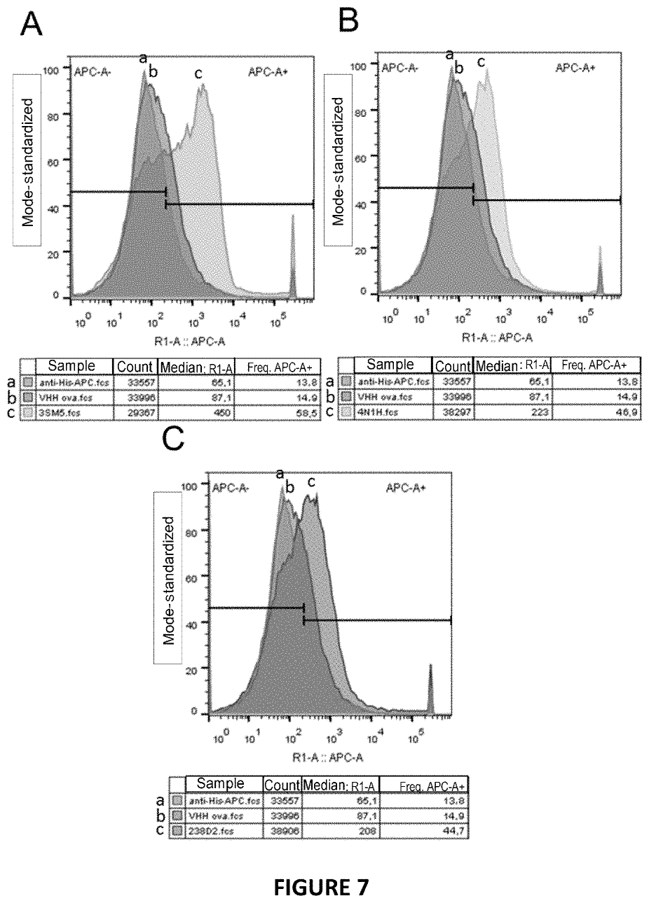

The cells were transiently transfected with the human CXCR4. Forty-eight hours after transfection, the cells were saturated for one hour in PBS-1% BSA, and exposed to the VHHs diluted to 1 μM in PBS-BSA for FIG. 6 and to 100 nM for FIG. 7.

For FIG. 6, after 2 washes, the cells were exposed to the anti-His revealing antibody coupled to allophycocyanin (APC) in PBS-BSA for 30 min at room temperature. The fluorescent signal was recorded using the FACScalibur (BD Biosciences, San Jose, Calif., USA).

For FIG. 7, after 2 washes, the cells were exposed to the anti-His revealing antibody coupled to allophycocyanin (APC) in PBS-BSA for 60 min at 4° C. The fluorescent signal was recorded using the MACSQuant Analyzer 10 (Miltenyi Biotech GmbH, Bergisch Gladbach, Germany).

At least two thousand events were measured and the peaks were compared to the signal of the anti-His-APC alone.

SDF1 Displacement

HEK293 cells were transiently transfected with CXCR4 coupled to a SNAP tag (Cisbio Bioassays, France). Forty-eight hours after transfection, the cells were coupled to the terbium ion according to the Lumi-4 terbium (Cisbio Bioassay) SNAP kit protocol. After several washes in tag-lite labeling medium (Cisbio Bioassays) buffer, 20 000 cells were distributed in a 384-well plate. The nano-antibodies are added at the indicated doses over 1 hour at 37° C. The SDF1 coupled to the d2 acceptor was added at the final concentration of 12 nM. After overnight incubation at 4° C., the plates were read using the Mithras² LB 943 Monochromator Multimode Reader (Berthold Technologies, Thoiry, France).

Epitope Mapping

The N-ter biotinylated peptides were synthesized by Genecust. In a 384-well plate, the peptides were deposited at the final concentration of 2 mM with the nano-antibodies at the final concentration of 2 μM in PBS-0.1% tween 20. After overnight incubation at 4° C., 2.35 μM streptavidin coupled to the d2 acceptor, and 50 nM of anti-His antibody coupled to the terbium donor were added. The plate was read 24 h later using the Mithras² LB 943 Monochromator Multimode Reader (Berthold Technologies, Thoiry, France). The results indicated correspond to the ratio between the signal emitted by the acceptor and the signal emitted by the donor.

Biolayer Interferometry (BLI)

The measurements were all carried out with the Octet RED96 System (Pall forte Bio, Fremont, Calif., USA), in the kinetic buffer from Pall Forte, at 30° C. and with stirring at 1000 rpm. The nano-antibodies 238D2 and 3SM5 coupled to the biotinylated AviTag were immobilized on streptavidin biosensors at 100 nM then left to stabilize for 120 seconds in the buffer. The binding of the Influenza A H1N1 (A/SOLOMON ISLANDS/3/2006) was measured at 50, 30, 20, 15, 10, 7.5 and 5 μg/ml for 5 minutes. A correction was carried out by subtracting the H1N1 binding to a non-relevant biotinylated protein. The results were analyzed with the Octet Software version 9.0 according to the 1:1 interaction model. An overall analysis was carried out of all the curves using the assumption that the dissociation is reversible. A single set of binding parameters results therefrom for all the concentrations used.

Immunoprecipitation

Ten μg of nano-antibodies coupled to the biotinylated AviTag and 10 μg of Influenza A H1N1 (A/SOLOMON ISLANDS/3/2006) were mixed in a final volume of 500 μl of TNET buffer (20 mM Tris HCl pH 8, 137 mM NaCl, 1% Nonidet P-40, 2 mM EDTA) and left overnight at 40° C. with stirring. Five hundred microliters of magnetic beads coupled to A and G proteins (Merck Millipore, armstadt, Germany) were equilibrated in TNET buffer before being loaded with 10 μl of anti-myc 9E10 antibody (Abcam, Cambridge, Mass., USA) overnight at 4° C. After 3 washes in TNET, the beads were exposed to the nano-antibody—H1N1 mixture for 2 h at 4° C. The beads were washed 3 times in TNET and suspended in 2× loading blue (0.02% bromophenol blue, 125 mM Tris HCl pH6.8, 20% Glycerol, 4% SDS, 10% β-ME). The protein supernatants were heated at 100° C. for 5 min, separated on SDS-PAGE and transferred to nitrocellulose membrane. The membranes were saturated in Tris Buffer Saline—0.01% Tween 20 (TBS-T)—3% BSA for 30 min at room temperature. The membranes were hybridized with an anti-His antibody (Anti-6×His tag antibody—ChIP Grade, Abcam, Cambridge, Mass., USA) itself revealed by the goat anti-rabbit secondary antibody IRDye 680 (Goat anti-Rabbit secondary antibody LI-COR, Lincoln, Nebr., USA). Simultaneously, the membranes were hybridized with streptavidin coupled to AlexaFluor. The membranes were scanned with Odyssey CLx (LI-COR, Lincoln, Nebr., USA).

1-13. (canceled)

14. A method making it possible to identify antibodies capable of binding to the same target, the method comprising:
providing a database comprising the characterization of at least 100 antibodies, each antibody being characterized by a simplified sequence and a secondary structure of the CDRs (complementarity-determining regions) CDR1, CDR2 and CDR3 for each variable domain of an antibody of the database, and;
the simplified sequence being a translated sequence in which each amino acid of the initial sequence of the CDRs is replaced by a code representing a category of amino acids, the categories being based on the physicochemical characteristics of the amino acids, the number of categories being between 4 and 10; and the categories being chosen from the size of the amino acids, their hydrophobicity, their polarity, their charge, their aromatic nature and combinations of these characteristics;
the secondary structure being a translated sequence in which each amino acid of the initial sequence of the CDRs is replaced by a code representing a type of secondary structure, the number of types of secondary structure being between 3 and 8 and the types of secondary structure being chosen from a 3₁₀ helix, an α helix, a π helix, a tight turn, a β strand, a residue in a β-bridge, a turn or an absence of secondary structure;
providing a simplified sequence and a secondary structure of the CDRs CDR1, CDR2 and CDR3 for a variable domain of a reference antibody;
calculating a score for similarity between the variable domain of the reference antibody and a variable domain of a test antibody from the database based on the simplified sequence of their CDRs and on the secondary structure of their CDRs; and
selecting the test antibody if the similarity score is such that it predicts that the test antibody and the reference antibody are capable of binding to the same target, the test antibody selected being referred to as similar antibody.

15. The method according to claim 14, characterized in that the method also comprises a step of in vitro validation of the binding capacity of the similar antibody to the target of the reference antibody or of the reference antibody to the target of the similar antibody.

16. The method according to claim 14, characterized in that the categories chosen are
small size for the amino acids A, G, S, T, C and P;
aromatic nature for the amino acids Y, F and W;
hydrophobicity for the amino acids I, L, F, M and V;
polarity for the amino acids N and Q;
positive charge for the amino acids H, K and R; and
negative charge for the amino acids D and E.

17. The method according to claim 14, characterized in that the types of secondary structure chosen are:
3₁₀ helix;
α helix;
π helix;
tight turn;
β strand;
residue in a β-bridge;
turn; and
absence of secondary structure.

18. The method according to claim 14, characterized in that the similarity score is calculated by identifying subsequences common to the CDRs of the reference antibody and of the test antibody.

19. The method according to claim 15, comprising a step of in vitro validation of the binding capacity of the similar antibody to the target of the reference antibody.

20. The method according to claim 19, wherein the validation step comprises providing or producing the similar antibody, bringing it into contact with the target of the reference antibody, and measuring the binding between the similar antibody and the target of the reference antibody.

21. The method according to claim 19, wherein the CDR1, CDR2 and CDR3 are grafted into a nano-antibody backbone.

22. The method according to claim 19, characterized in that the similar antibody is selected if the binding capacity of the similar antibody to the target of the reference antibody is validated in vitro.

23. The method according to claim 15, comprising a step of in vitro validation of the binding capacity of the reference antibody to the target of the similar antibody.

24. The method according to claim 23, wherein the validation step comprises producing or providing the reference antibody, bringing it into contact with the target of the similar antibody, and measuring the binding between the reference antibody and the target of the similar antibody.

25. The method according to claim 23, characterized in that the reference antibody is selected if the binding capacity of the reference antibody to the target of the similar antibody is validated in vitro.

26. The method according to claim 14, characterized in that the antibodies are nano-antibodies.


SEQUENCE LISTING (45 sequences)

SEQ ID NO: 1 - protein, 26 aa, artificial sequence
  FR1
MEVQLQESGGGLVQAGASLKLSCAAS

SEQ ID NO: 2 - protein, 26 aa, artificial sequence
  FR1
MEVQLVQSGAEVKKPGASVKVSCKAS

SEQ ID NO: 3 - protein, 17 aa, artificial sequence
  FR2
MGWFRQAPGKEREFVAA

SEQ ID NO: 4 - protein, 18 aa, artificial sequence
  FR2
HINWVRQAPGQGLEWMGW

SEQ ID NO: 5 - protein, 39 aa, artificial sequence
  FR3
TKYADSVKGRFAISRDNDKNTVWLRMNSLKPEDTAVYYC

SEQ ID NO: 6 - protein, 38 aa, artificial sequence
  FR3
NYAQKFQGWVTMTRDTAISTAYMEVNGLKSDDTAVYYC

SEQ ID NO: 7 - protein, 11 aa, artificial sequence
  FR4
WGQGTQVTVSS

SEQ ID NO: 8 - protein, 128 aa, artificial sequence
  4N1H
MEVQLQESGGGLVQAGASLKLSCAASGRTFSSYAMGWFRQAPGKEREFVAAISRSGGDTKYADSVKGRFAISRDNDKNTVWLRMNSLKPEDTAVYYCAATTYASLSDTYIGEHIYDDWGQGTQVTVSS

SEQ ID NO: 9 - protein, 139 aa, artificial sequence
  4N1H - myc tag
MEVQLQESGGGLVQAGASLKLSCAASGRTFSSYAMGWFRQAPGKEREFVAAISRSGGDTKYADSVKGRFAISRDNDKNTVWLRMNSLKPEDTAVYYCAATTYASLSDTYIGEHIYDDWGQGTQVTVSSEQKLISEEDLE

SEQ ID NO: 10 - protein, 145 aa, artificial sequence
  4N1H - his tag
MEVQLQESGGGLVQAGASLKLSCAASGRTFSSYAMGWFRQAPGKEREFVAAISRSGGDTKYADSVKGRFAISRDNDKNTVWLRMNSLKPEDTAVYYCAATTYASLSDTYIGEHIYDDWGQGTQVTVSSEQKLISEEDLEHHHHHH

SEQ ID NO: 11 - protein, 127 aa, artificial sequence
  3SM5
MEVQLVQSGAEVKKPGASVKVSCKASGYTFTDYHINWVRQAPGQGLEWMGWIHPNSGDTNYAQKFQGWVTMTRDTAISTAYMEVNGLKSDDTAVYYCARGGLEPRSVDYYYYGMDVWGQGTTVTVSS

SEQ ID NO: 12 - protein, 138 aa, artificial sequence
  3SM5 - myc tag
MEVQLVQSGAEVKKPGASVKVSCKASGYTFTDYHINWVRQAPGQGLEWMGWIHPNSGDTNYAQKFQGWVTMTRDTAISTAYMEVNGLKSDDTAVYYCARGGLEPRSVDYYYYGMDVWGQGTTVTVSSEQKLISEEDLE

SEQ ID NO: 13 - protein, 144 aa, artificial sequence
  3SM5 - his tag
MEVQLVQSGAEVKKPGASVKVSCKASGYTFTDYHINWVRQAPGQGLEWMGWIHPNSGDTNYAQKFQGWVTMTRDTAISTAYMEVNGLKSDDTAVYYCARGGLEPRSVDYYYYGMDVWGQGTTVTVSSEQKLISEEDLEHHHHHH

SEQ ID NO: 14 - protein, 8 aa, artificial sequence
  CDR1 238D2
GFTFSSYA

SEQ ID NO: 15 - protein, 8 aa, artificial sequence
  CDR2 238D2
IKSSGDST

SEQ ID NO: 16 - protein, 15 aa, artificial sequence
  CDR3 238D2
AKSRVSRTGLYTYDN

SEQ ID NO: 17 - protein, 8 aa, artificial sequence
  CDR1 238D4
GRTFNNYA

SEQ ID NO: 18 - protein, 12 aa, artificial sequence
  CDR2 238D4
ITRSGVRSGVSA

SEQ ID NO: 19 - protein, 17 aa, artificial sequence
  CDR3 238D4
AASAIGSGALRRFEYDY

SEQ ID NO: 20 - protein, 7 aa, artificial sequence
  CDR1 4N1H
GRTFSSY

SEQ ID NO: 21 - protein, 6 aa, artificial sequence
  CDR2 4N1H
SRSGGD

SEQ ID NO: 22 - protein, 18 aa, artificial sequence
  CDR3 4N1H
TTYASLSDTYIGEHIYDD

SEQ ID NO: 23 - protein, 7 aa, artificial sequence
  CDR1 3SM5
GYTFTDY

SEQ ID NO: 24 - protein, 6 aa, artificial sequence
  CDR2 3SM5
HPNSGD

SEQ ID NO: 25 - protein, 17 aa, artificial sequence
  CDR3 3SM5
GGLEPRSVDYYYYGMDV

SEQ ID NO: 26 - protein, 8 aa, artificial sequence
  Simplified CDR1 238D2
  MISC_FEATURE (1)..(1): Xaa = A, G, S, T, C or P
  MISC_FEATURE (2)..(2): Xaa = Y, F or W
  MISC_FEATURE (3)..(3): Xaa = A, G, S, T, C or P
  MISC_FEATURE (4)..(4): Xaa = Y, F or W
  MISC_FEATURE (5)..(6): Xaa = A, G, S, T, C or P
  MISC_FEATURE (7)..(7): Xaa = Y, F or W
  MISC_FEATURE (8)..(8): Xaa = A, G, S, T, C or P
XXXXXXXX

SEQ ID NO: 27 - protein, 8 aa, artificial sequence
  Simplified CDR2 238D2
  MISC_FEATURE (1)..(1): Xaa = I, L, F, M or V
  MISC_FEATURE (2)..(2): Xaa = H, K or R
  MISC_FEATURE (3)..(5): Xaa = A, G, S, T, C or P
  MISC_FEATURE (6)..(6): Xaa = D or E
  MISC_FEATURE (7)..(8): Xaa = A, G, S, T, C or P
XXXXXXXX

SEQ ID NO: 28 - protein, 15 aa, artificial sequence
  Simplified CDR3 238D2
  MISC_FEATURE (1)..(1): Xaa = A, G, S, T, C or P
  MISC_FEATURE (2)..(2): Xaa = H, K or R
  MISC_FEATURE (3)..(3): Xaa = A, G, S, T, C or P
  MISC_FEATURE (4)..(4): Xaa = H, K or R
  MISC_FEATURE (5)..(5): Xaa = I, L, F, M or V
  MISC_FEATURE (6)..(6): Xaa = A, G, S, T, C or P
  MISC_FEATURE (6)..(6): Xaa = H, K or R
  misc_feature (7)..(7): Xaa can be any naturally occurring amino acid
  MISC_FEATURE (8)..(9): Xaa = A, G, S, T, C or P
  MISC_FEATURE (10)..(10): Xaa = I, L, F, M or V
  MISC_FEATURE (11)..(11): Xaa = Y, F or W
  MISC_FEATURE (12)..(12): Xaa = A, G, S, T, C or P
  MISC_FEATURE (13)..(13): Xaa = Y, F or W
  MISC_FEATURE (14)..(14): Xaa = D or E
  MISC_FEATURE (15)..(15): Xaa = N or Q
XXXXXXXXXXXXXXX

SEQ ID NO: 29 - protein, 8 aa, artificial sequence
  Simplified CDR1 238D4
  MISC_FEATURE (1)..(1): Xaa = A, G, S, T, C or P
  MISC_FEATURE (2)..(2): Xaa = H, K or R
  MISC_FEATURE (3)..(3): Xaa = A, G, S, T, C or P
  MISC_FEATURE (4)..(4): Xaa = Y, F or W
  MISC_FEATURE (5)..(6): Xaa = N or Q
  MISC_FEATURE (7)..(7): Xaa = Y, F or W
  MISC_FEATURE (8)..(8): Xaa = A, G, S, T, C or P
XXXXXXXX

SEQ ID NO: 30 - protein, 12 aa, artificial sequence
  Simplified CDR2 238D4
  MISC_FEATURE (1)..(1): Xaa = I, L, F, M or V
  MISC_FEATURE (2)..(2): Xaa = A, G, S, T, C or P
  MISC_FEATURE (3)..(3): Xaa = H, K or R
  MISC_FEATURE (4)..(5): Xaa = A, G, S, T, C or P
  MISC_FEATURE (6)..(6): Xaa = I, L, F, M or V
  MISC_FEATURE (7)..(7): Xaa = H, K or R
  MISC_FEATURE (8)..(9): Xaa = A, G, S, T, C or P
  MISC_FEATURE (10)..(10): Xaa = I, L, F, M or V
  MISC_FEATURE (11)..(12): Xaa = A, G, S, T, C or P
XXXXXXXXXXXX

SEQ ID NO: 31 - protein, 17 aa, artificial sequence
  Simplified CDR3 238D4
  MISC_FEATURE (1)..(4): Xaa = A, G, S, T, C or P
  MISC_FEATURE (5)..(5): Xaa = I, L, F, M or V
  MISC_FEATURE (6)..(9): Xaa = A, G, S, T, C or P
  MISC_FEATURE (10)..(10): Xaa = I, L, F, M or V
  MISC_FEATURE (11)..(12): Xaa = H, K or R
  MISC_FEATURE (13)..(13): Xaa = Y, F or W
  MISC_FEATURE (14)..(14): Xaa = D or E
  MISC_FEATURE (15)..(15): Xaa = Y, F or W
  MISC_FEATURE (16)..(16): Xaa = D or E
  MISC_FEATURE (17)..(17): Xaa = Y, F or W
XXXXXXXXXXXXXXXXX

SEQ ID NO: 32 - protein, 7 aa, artificial sequence
  Simplified CDR1 4N1H
  MISC_FEATURE (1)..(1): Xaa = A, G, S, T, C or P
  MISC_FEATURE (2)..(2): Xaa = H, K or R
  MISC_FEATURE (3)..(3): Xaa = A, G, S, T, C or P
  MISC_FEATURE (4)..(4): Xaa = Y, F or W
  MISC_FEATURE (5)..(6): Xaa = A, G, S, T, C or P
  MISC_FEATURE (7)..(7): Xaa = Y, F or W
XXXXXXX

SEQ ID NO: 33 - protein, 6 aa, artificial sequence
  Simplified CDR2 4N1H
  MISC_FEATURE (1)..(1): Xaa = A, G, S, T, C or P
  MISC_FEATURE (2)..(2): Xaa = H, K or R
  MISC_FEATURE (3)..(5): Xaa = A, G, S, T, C or P
  MISC_FEATURE (6)..(6): Xaa = D or E
XXXXXX

SEQ ID NO: 34 - protein, 18 aa, artificial sequence
  Simplified CDR3 4N1H
  MISC_FEATURE (1)..(2): Xaa = A, G, S, T, C or P
  MISC_FEATURE (3)..(3): Xaa = Y, F or W
  MISC_FEATURE (4)..(5): Xaa = A, G, S, T, C or P
  MISC_FEATURE (6)..(6): Xaa = I, L, F, M or V
  MISC_FEATURE (7)..(7): Xaa = A, G, S, T, C or P
  MISC_FEATURE (8)..(8): Xaa = D or E
  MISC_FEATURE (9)..(9): Xaa = A, G, S, T, C or P
  MISC_FEATURE (10)..(10): Xaa = Y, F or W
  MISC_FEATURE (11)..(11): Xaa = I, L, F, M or V
  MISC_FEATURE (12)..(12): Xaa = A, G, S, T, C or P
  MISC_FEATURE (13)..(13): Xaa = D or E
  MISC_FEATURE (14)..(14): Xaa = H, K or R
  MISC_FEATURE (15)..(15): Xaa = I, L, F, M or V
  MISC_FEATURE (16)..(16): Xaa = Y, F or W
  MISC_FEATURE (17)..(18): Xaa = D or E
XXXXXXXXXXXXXXXXXX

SEQ ID NO: 35 - protein, 7 aa, artificial sequence
  Simplified CDR1 3SM5
  MISC_FEATURE (1)..(1): Xaa = A, G, S, T, C or P
  MISC_FEATURE (2)..(2): Xaa = Y, F or W
  MISC_FEATURE (3)..(3): Xaa = A, G, S, T, C or P
  MISC_FEATURE (4)..(4): Xaa = Y, F or W
  MISC_FEATURE (5)..(5): Xaa = A, G, S, T, C or P
  MISC_FEATURE (6)..(6): Xaa = D or E
  MISC_FEATURE (7)..(7): Xaa = Y, F or W
XXXXXXX

SEQ ID NO: 36 - protein, 6 aa, artificial sequence
  Simplified CDR2 3SM5
  MISC_FEATURE (1)..(1): Xaa = H, K or R
  MISC_FEATURE (2)..(2): Xaa = A, G, S, T, C or P
  MISC_FEATURE (3)..(3): Xaa = N or Q
  MISC_FEATURE (4)..(5): Xaa = A, G, S, T, C or P
  misc_feature (6)..(6): Xaa can be any naturally occurring amino acid
  MISC_FEATURE (7)..(7): Xaa = D or E
XXXXXX

SEQ ID NO: 37 - protein, 17 aa, artificial sequence
  Simplified CDR3 3SM5
  MISC_FEATURE (1)..(2): Xaa = A, G, S, T, C or P
  MISC_FEATURE (3)..(3): Xaa = I, L, F, M or V
  MISC_FEATURE (4)..(4): Xaa = D or E
  MISC_FEATURE (5)..(5): Xaa = A, G, S, T, C or P
  MISC_FEATURE (6)..(6): Xaa = H, K or R
  MISC_FEATURE (7)..(7): Xaa = A, G, S, T, C or P
  MISC_FEATURE (8)..(8): Xaa = I, L, F, M or V
  MISC_FEATURE (9)..(9): Xaa = D or E
  MISC_FEATURE (10)..(13): Xaa = Y, F or W
  MISC_FEATURE (14)..(14): Xaa = A, G, S, T, C or P
  MISC_FEATURE (15)..(15): Xaa = I, L, F, M or V
  MISC_FEATURE (16)..(16): Xaa = D or E
  MISC_FEATURE (17)..(17): Xaa = I, L, F, M or V
XXXXXXXXXXXXXXXXX

SEQ ID NO: 38 - protein, 177 aa, artificial sequence
  AviTag - 3SM5 - myc tag
MGSSHHHHHHSSGLVPRGSHMSGLNDIFEAQKIEWHEDPNSEVQLVQSGAEVKKPGASVKVSCKASGYTFTDYHINWVRQAPGQGLEWMGWIHPNSGDTNYAQKFQGWVTMTRDTAISTAYMEVNGLKSDDTAVYYCARGGLEPRSVDYYYYGMDVWGQGTTVTVSSEQKLISEEDL

SEQ ID NO: 39 - protein, 134 aa, artificial sequence
  238D2- myc tag
MEVQLVESGGGLVQTGGSLRLSCAASGFTFSSYAMSWVRQAPGKGLEWVSGIKSSGDSTRYAGSVKGRFTISRDNAKNMLYLQMYSLKPEDTAVYYCAKSRVSRTGLYTYDNRGQGTQVTVSSEQKLISEEDLE

SEQ ID NO: 40 - protein, 140 aa, artificial sequence
  238D2- his tag
MEVQLVESGGGLVQTGGSLRLSCAASGFTFSSYAMSWVRQAPGKGLEWVSGIKSSGDSTRYAGSVKGRFTISRDNAKNMLYLQMYSLKPEDTAVYYCAKSRVSRTGLYTYDNRGQGTQVTVSSEQKLISEEDLEHHHHHH

SEQ ID NO: 41 - protein, 173 aa, artificial sequence
  AviTag - 238D2 - myc tag
MGSSHHHHHHSSGLVPRGSHMSGLNDIFEAQKIEWHEDPNSEVQLVESGGGLVQTGGSLRLSCAASGFTFSSYAMSWVRQAPGKGLEWVSGIKSSGDSTRYAGSVKGRFTISRDNAKNMLYLQMYSLKPEDTAVYYCAKSRVSRTGLYTYDNRGQGTQVTVSSEQKLISEEDL

SEQ ID NO: 42 - protein, 140 aa, artificial sequence
  238D4- myc tag
MEVQLMESGGGLVQAGGSLRLSCAASGRTFNNYAMGWFRRAPGKEREFVAAITRSGVRSGVSAIYGDSVKDRFTISRDNAKNTLYLQMNSLKPEDTAVYTCAASAIGSGALRRFEYDYSGQGTQVTVSSEQKLISEEDLE

SEQ ID NO: 43 - protein, 146 aa, artificial sequence
  238D4- his tag
MEVQLMESGGGLVQAGGSLRLSCAASGRTFNNYAMGWFRRAPGKEREFVAAITRSGVRSGVSAIYGDSVKDRFTISRDNAKNTLYLQMNSLKPEDTAVYTCAASAIGSGALRRFEYDYSGQGTQVTVSSEQKLISEEDLEHHHHHH

SEQ ID NO: 44 - protein, 182 aa, artificial sequence
  Ovalbumin - his tag
MGSSHHHHHHSSGLVPRGSHMASMTGGQQMGRGSEFELRRQACGRTRAPPPPPLRSGCEVQLQESGGSGQAGGSLRLSCAASGDTVRTMAWFRQAPGQEREGVAGFNLPISRPYYADGMKARFTISGDKSKNTVTLQMDNLAPEDTANYYCAATRYTLDLSSRIFQGDFDHWGHGTQVTVSS

SEQ ID NO: 45 - protein, 498 aa, Homo sapiens
MEGISIYTSDNYTEEMGSGDYDSMKEPCFREENANFNKIFLPTIYSIIFLTGIVGNGLVILVMGYQKKLRSMTDKYRLHLSVADLLFVITLPFWAVDAVANWYFGNFLCKAVHVIYTVNLYSSVWILAFISLDRYLAIVHATNSQRPRKLLAEKVVYVGVWIPALLLTIPDFIFANVSEADDRYICDRFYPNDLWVVVFQFQHIMVGLILPGIVILSCYCIIISKLSHSGSNIFEMLRIDEGLRLKIYKDTEGYYTIGIGHLLTKSPSLNAAKSELDKAIGRNTNGVITKDEAEKLFNQDVDAAVRGILRNAKLKPVYDSLDAVRRAALINMVFQMGETGVAGFTNSLRMLQQKRWDEAAVNLAKSRWYNQTPNRAKRVITTFRTGTWDAYGSKGHQKRKALKTTVILILAFFACWLPYYIGISIDSFILLEIIKQGCEFENTVHKWISITEALAFFHCCLNPILYAFLGAKFKTSAQHALTSVSRGSSGRPLEVLFQ